(19) United States
(12) Patent Application Publication
(10) Pub. No.: US 2008/0045843 A1
(43) Pub. Date: Feb. 21, 2008

(54) Via-Human-Body Information Transmission System and Transmitter-Receiver

(76) Inventor: Tomoharu Tsuji, Chiba-shi (JP)

Correspondence Address: Bruce L Adams;Adams & Wilks, 17 Batter Place, Suite 1231, New York, NY, 10004, US

(21) Appl. No.: 11/663,104

(22) PCT Filed: Dec. 7, 2005
(86) PCT No.: PCT/JP05/22423

§ 371 Date: Apr. 27, 2007

(30) Foreign Application Priority Data
Aug. 12, 2004 (JP) 2004-355514

(51) Int. Cl. A61B 5/02

(52) U.S. Cl. 600/481

(57) ABSTRACT

In a via-human-body information transmission system, there is made so as to be capable of more stably transmitting a signal.

An electrode (104) of a transmitter-receiver (102) contacting with or capacitance-connected to a human body (101) and an electrode (108) of a receiver (106) contacting with or capacitance-connected to the human body (101) are electrically connected through the human body (101) (mainly, a chest part and a left arm). An electrode (105) of the transmitter-receiver (102) contacting with or capacitance-connected to the human body (101) and an electrode (109), of the receiver (106), having been disposed while being directed to an outside of the human body (101) are electrically connected by a capacitance connection. A communication between the transmitter-receiver (102) and the receiver (106) is performed through a path passing the electrodes (104, 105, 108, 109) and the human body (101) and a path by the capacitance connection.

### TECHNICAL FIELD

The present invention relates to a via-human-body information transmission system performing an information transmission through a human body, and a transmitter-receiver.

### BACKGROUND ART

In recent years, there are eagerly proposed communications having utilized the human body as a transmission line (for example, refer to Patent Documents 1-3).

FIG. 6 is a system constitution view of the via-human-body information transmission system having been described in the Patent Document 1. In FIG. 6, a transmitter 402 is worn to one arm of a human body 401, and a receiver 405 is worn to the other arm. One electrode 403 of the transmitter 402 contacts with or is capacitance-connected to the human body 401, and the other electrode 404 is attached while being directed to an outside of the human body 401. Further, one electrode 406 of the receiver 405 contacts with or is capacitance-connected to the human body 401, and the other electrode 407 is attached while being directed to the outside of the human body 401.

In a signal line side, the transmitter 402 is connected to the receiver 405 through the electrode 403, the human body 401 and the electrode 406. Further, in a reference potential line (e.g., ground potential) side, the receiver 405 is connected to the transmitter 402 by such paths by the capacitance connection as shown by broken lines through the electrode 407, a reference potential point (e.g., ground) and the electrode 404.

In the via-human-body information transmission system having been constituted like the above, a signal having been transmitted from the transmitter 402 is transmitted to the receiver 405 through the electrode 403, the human body 401 and the electrode 406. By doing like this, although it is possible to transmit the signal having been outputted from the transmitter 402 to the receiver 405, there are problems that a signal transmission operation is unstable because a length, through which the capacitance connection is utilizes, is long, and that a miniaturization is difficult because it is necessary to increase areas of the electrodes 403, 404, 406, 407.

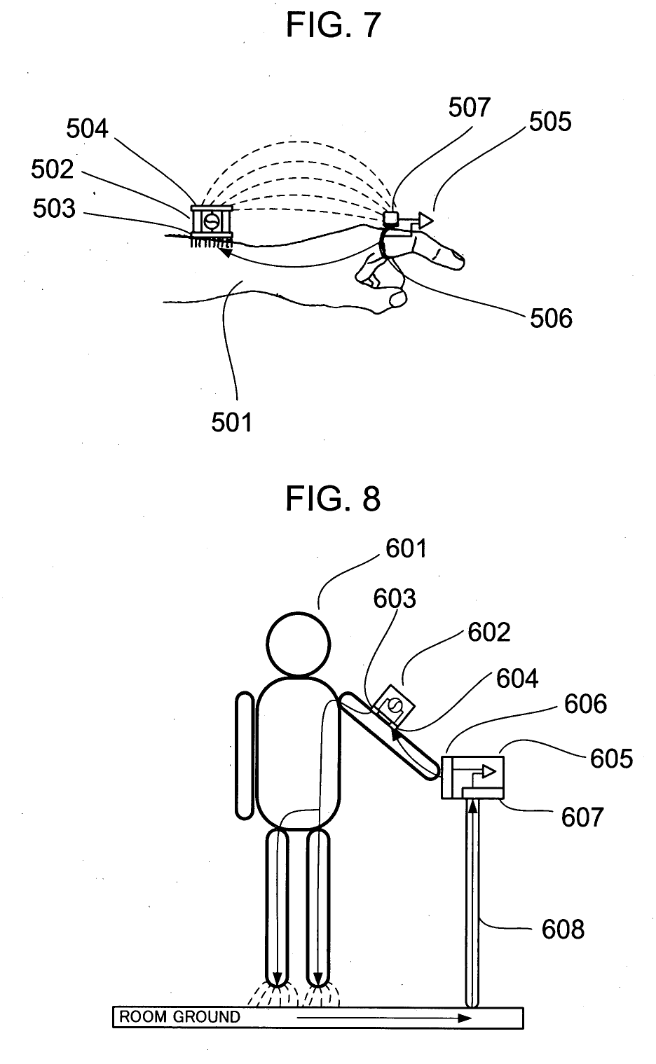

FIG. 7 is a system constitution view of the via-human-body information transmission system having been described in the Patent Document 2. In FIG. 7, a transmitter 502 is attached to an arm of a human body 501, and a receiver 505 is attached to a finger. One electrode 503 of the transmitter 502 contacts with or is capacitance-connected to the human body 501, and the other electrode 504 is attached while being directed to an outside of the human body 501. Further, one electrode 506 of the receiver 505 contacts with or is capacitance-connected to the human body 501, and the other electrode 507 is attached while being directed to the outside of the human body 501.

In the signal line side, the transmitter 502 is electrically connected to the receiver 505 by such paths of the capacitance connection as shown by broken lines through the electrode 504 and the electrode 507. Further, in the reference potential line (e.g., ground potential) side, the receiver 505 is connected to the transmitter 502 through the electrode 506, the human body 501 and the electrode 503.

In the via-human-body information transmission system having been constituted like the above, a signal having been transmitted from the transmitter 502 is transmitted to the receiver 505 by the paths by the capacitance connection through the electrode 504 and the electrode 507. By doing like this, although it is possible to transmit the signal having been outputted from the transmitter 502 to the receiver 505, since it is a constitution in which both of the electrode 504 and the electrode 507 have been attached while being directed to the outside of the human body 501, there is a problem that an intensity of the capacitance connection changes by a person's motion and thus a signal transmission operation becomes unstable. Further, there is a problem that a distance between the transmitter 502 and the receiver 505 cannot be made long.

FIG. 8 is a system constitution view of the via-human-body information transmission system having been described in the Patent Document 3. In FIG. 8, a first communication equipment 602 is worn to one arm of a human body 601. A second communication equipment 605 is attached to a supporting column 608 of a conductor, which has been connected to the reference potential (e.g., ground potential). Two electrodes 603, 604 of the communication equipment 602 contact with or are capacitance-connected to the human body 601. Further, one electrode 606 of the communication equipment 605 is attached while being directed to a space, and the other electrode 607 is attached to the supporting column 608 so as to be electrically connected thereto.

In the signal line side, the communication equipment 605 is connected to the communication equipment 602 through the electrode 606, the human body 601 and the electrode 604. Further, in a reference potential line (e.g., ground potential) side, the communication equipment 602 is connected to the communication equipment 605 through the electrode 603, the human body 601, paths by the capacitance connection shown by broken lines, the reference potential (ground potential), the supporting column 608 and the electrode 607.

In the via-human-body information transmission system having been constituted like the above, if a fingertip of the human body 601 is contacted with the electrode 606, the communication equipment 605 transmits an information to the communication equipment 602 through the electrode 606, the human body 601 and the electrode 604. Incidentally, at this time, although the two electrodes 603, 604 are attached so as to contact with or be capacitance-connected to the human body 601, between the electrode 603 and the electrode 604, since there is no short circuit and there exists an impedance of an intensity of certain degree, it is possible to transmit a signal having been outputted from the communication equipment 605 to the communication equipment 602.

However, since the system of FIG. 8 is a system whose object is a use as an ID tag, it is constituted so as to be used with only one communication equipment being worn to the human body, and thus there is not considered such a constitution that plural communication equipment are worn to the human body and the communication is performed between the plural communication equipment.

[Patent Document 1]

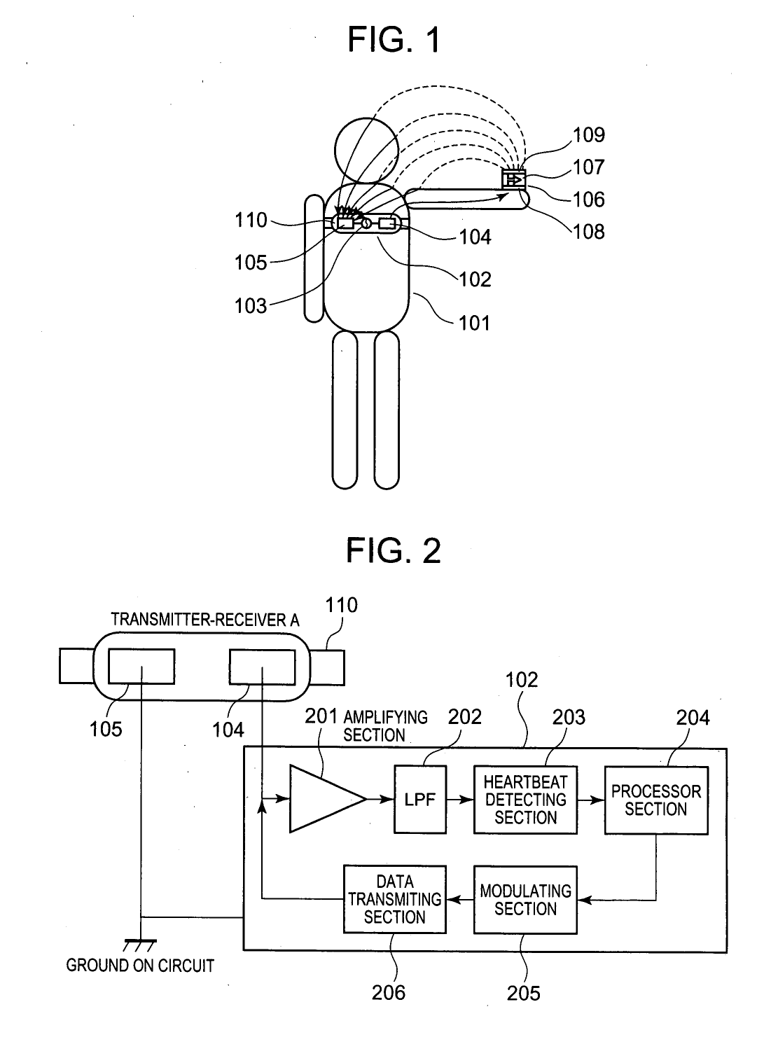

U.S. Pat. No. 5,914,701 Gazette, column 1, line 60-column 6, line 37, FIG. 1-FIG. 4

[Patent Document 2]

Japanese Patent No. 3425347 Gazette, columns [0034]-[0046], FIG. 1-FIG. 3

[Patent Document 3]

JP-A-2001-308803 Gazette, columns [0019]-[0030], FIG. 1-FIG. 6

### DISCLOSURE OF THE INVENTION

### Problems that the Invention is to Solve

Problems of the present invention are to constitute, in the via-human-body information transmission system performing the information transmission between plural communication means through the human body, so as to be used while attaching the plural communication means to the human body, and to make such that the information transmission can be more stably performed.

Further, a problem of the present invention is to simplify a constitution in the via-human-body information transmission system.

Further, a problem of the present invention is to provide a transmitter-receiver suitable for constructing the via-human-body information transmission system.

### Means for Solving the Problems

According to the present invention, there is provided a via-human-body information transmission system performing an information transmission between plural communication means through a human body, possessing a first communication means which has first and second electrodes contacting with or capacitance-connected to the human body and which is used while being attached to the human body, and a second communication means which has a third electrode contacting with or capacitance-connected to the human body and a fourth electrode having been disposed while being directed to an outside of the human body and which is used while being attached to the human body, characterized in that a communication is performed between the first and second communication means by the facts that the first electrode and the third elect-rode are electrically connected through the human body, and that the second electrode and the fourth electrode are electrically connected by a capacitance connection.

The first communication means which has the first and second electrodes contacting with or capacitance-connected to the human body and the second communication means which has the third electrode contacting with or capacitance-connected to the human body and the fourth electrode having been disposed while being directed to the outside of the human body are used while being attached to the human body. The first electrode and the third electrode are electrically connected through the human body, the second electrode and the fourth electrode are electrically connected by the capacitance connection and, by this, the communication between the first and second communication means is performed.

Here, there may be constituted such that an organism information of the human body is transmitted between the first communication means and the second communication means, and the first and second electrodes of the first communication means are used both for an electrode detecting the organism information of the human body and for an electrode performing a transmission of the organism information.

Further, there may be constituted such that a frequency of a carrier signal used in the communication between the first communication means and the second communication means is set to a frequency different from a frequency of the organism information of the human body.

Further, there may be constituted such that the frequency of the carrier signal is a frequency within a range of 5 MHz-45 MHz.

Further, there may be constituted such that the organism information is an information of a heartbeat.

Further, there may be constituted such that the first communication means is attached to the human body by a chest band.

Further, there may be constituted such that the first communication means has a filter through which a signal of a predetermined frequency in a signal having been received by the first electrode passes and which outputs it, a heartbeat detecting section detecting a heartbeat signal from an output signal of the filter, a first processor section calculating a heartbeat number on the basis of the heartbeat signal having been detected in the heartbeat detecting section to thereby output a heartbeat number data signal denoting the heartbeat number, a modulating section modulating and outputting the heartbeat number data signal, and a data transmitting section outputting the heartbeat number data signal having been modulated in the modulating section, and the second communication means has a demodulating section demodulating the heartbeat number data signal having been received by the third electrode, a storage means, a display section, and a second processor section which stores a heartbeat number data having been obtained from the heartbeat number data signal having been demodulated in the demodulating section to the storage means and which display-processes the heartbeat number to the display section.

Further, according to the present invention there is provided a transmitter-receiver characterized by having a filter through which a signal of a predetermined frequency in a signal having been received by an electrode contacting with or capacitance-connected to a human body passes and which outputs it, a heartbeat detecting section detecting a heartbeat signal from an output signal of the filter, a processor section calculating a heartbeat number on the basis of the heartbeat signal having been detected in the heartbeat detecting section to thereby output a heartbeat number data signal denoting the heartbeat number, a modulating section modulating and outputting the heartbeat number data signal, and a data transmitting section outputting the heartbeat number data signal having been modulated in the modulating section from the first electrode.

The filter causes the signal of the predetermined frequency in the signal having been received by the electrode contacting with or capacitance-connected to the human body to passe and outputs it. The heartbeat detecting section detects the heartbeat signal from the output signal of the filter. The processor section calculates the heartbeat number on the basis of the heartbeat signal having been detected in the heartbeat detecting section to thereby output the heartbeat number data signal denoting the heartbeat number. The modulating section modulates and outputs the heartbeat number data signal. The data transmitting section outputs the heartbeat number data signal having been modulated in the modulating section from the electrode.

Here, there may be constituted such that a frequency of a carrier signal that the modulating section uses for modulating the heartbeat number data signal is a frequency within a range of 5 MHz-45 MHz.

### ADVANTAGES OF THE INVENTION

According to the via-human-body information transmission system concerned with the present invention, since it becomes possible to shorten a distance through which the capacitance connection is performed, it becomes possible to more stabilize an information transmission operation.

Further, by the fact that the electrode used for the detection of the organism information and the electrode used for the communication are commonly used, it becomes unnecessary to provide a new communication means, so that it becomes possible to inexpensively constitute by a simple constitution.

Further, according to the present invention, it becomes possible to provide a transmitter-receiver suitable for constructing the via-human-body information transmission system.

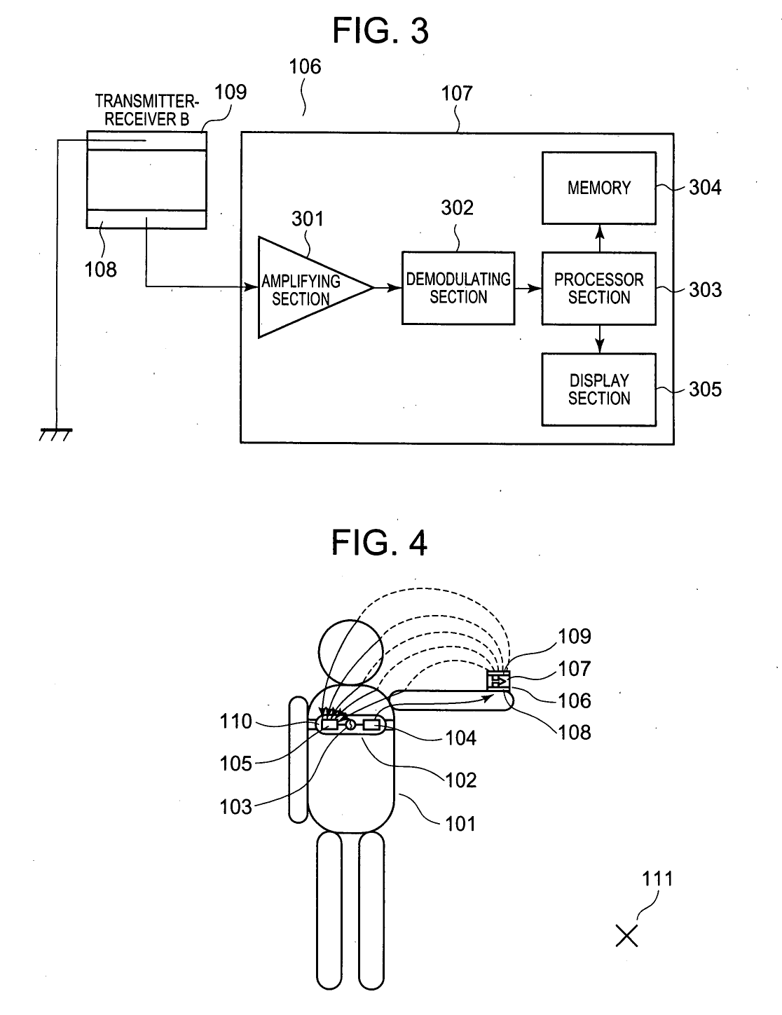

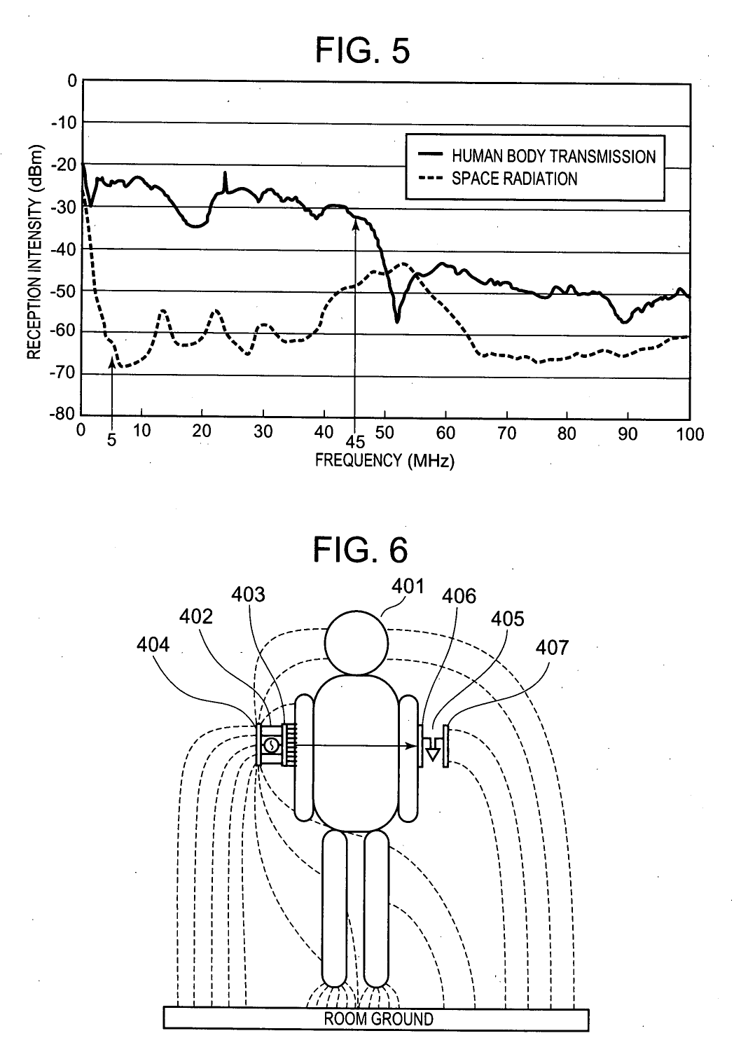

### BRIEF DESCRIPTION OF THE DRAWINGS

[FIG. 1] It is a system constitution view of a via-human-body information transmission system concerned with an embodiment of the present invention.

[FIG. 2] It is a detailed block diagram of a transmitter-receiver used in the via-human-body information transmission system concerned with the embodiment of the present invention.

[FIG. 3] It is a detailed block diagram of a receiver used in the via-human-body information transmission system concerned with the embodiment of the present invention.

[FIG. 4] It is a view when measuring a characteristic of the via-human-body information transmission system concerned with the embodiment of the present invention.

[FIG. 5] It is a characteristic diagram showing the characteristic of the via-human-body information transmission system concerned with the embodiment of the present invention.

[FIG. 6] It is a system constitution view of a conventional via-human-body information transmission system.

[FIG. 7] It is a system constitution view of a conventional via-human-body information transmission system.

[FIG. 8] It is a system constitution view of a conventional via-human-body information transmission system.

### DESCRIPTION OF REFERENCE NUMERALS

101 . . . human body
102 . . . transmitter-receiver as first communication means
103 . . . transmitter-receiver main body
104 . . . first electrode
105 . . . second electrode
106 . . . receiver as second communication means
107 . . . receiver main body
108 . . . third electrode
109 . . . fourth electrode
110 . . . chest belt
111 . . . measuring point
201, 301 . . . amplifying section
202 . . . filter
203 . . . heartbeat detecting section
204 . . . first processor section
205 . . . modulating section
206 . . . data transmitting section
302 . . . demodulating section
303 . . . second processor section
304 . . . memory as storage means
305 . . . display section as display means

### BEST MODE FOR CARRYING OUT THE INVENTION

Hereunder, there are explained about a via-human-body information transmission system and a transmitter-receiver, which are concerned with an embodiment of the present invention.

FIG. 1 is a system constitution view of the via-human-body information transmission system concerned with the embodiment of the present invention, and shows an example of an ictometer system as the via-human-body information transmission system.

Further, FIG. 2 is a detailed block diagram of a transmitter-receiver 102, and FIG. 3 is a detailed block diagram of a receiver 106. Incidentally, in FIG. 1-FIG. 3, the same reference numeral is applied to the same portion.

In FIG. 1-FIG. 3, the transmitter-receiver 102 as a first communication means and the receiver 106 as a second communication means are both used while being attached to a human body 101. The transmitter-receiver 102 is attached to a chest part of the human body 101 by a chest band 110. The receiver 106 is attached to one arm (e.g., wrist of a left arm) of the human body 101.

The transmitter-receiver 102 has a transmitter-receiver main body 103, a first electrode 104 contacting with or capacitance-connected to the human body 101, and a second electrode 105 contacting with or capacitance-connected to the human body 101. Further, the receiver 106 has a receiver main body 107, a third electrode 108 contacting with or capacitance-connected to the human body 101, and a fourth electrode 109 having been disposed while being directed to an outside (opposite side to the human body 101) of the human body 101.

The first electrode 104 and the third electrode 108 are electrically connected through the human body 101 (mainly, the chest part and the left arm that is the arm to which the receiver 106 has been attached), and the second electrode 105 and the fourth electrode 109 are electrically connected by a capacitance connection as shown by broken lines. By this, there is constituted such that a communication is possible between the transmitter-receiver 102 and the receiver 106.

That is, in the signal line side, the transmitter-receiver 102 is connected to the receiver 106 through the electrode 104, the human body 101 and the electrode 108. Further, in the reference potential line (e.g., ground potential) side, the receiver 106 is connected to the transmitter-receiver 102 by the paths by such a capacitance connection as shown by the broken lines through the electrode 109, the human body 101 and the electrode 105.

Even in any case of a case where the electrode 104 and the electrode 105 have been attached so as to contact with the human body, and a case where they have been attached so as to be capacitance-connected to the human body, between the electrode 104 and the electrode 105, since there is no short circuit and there exists the impedance, a signal transmission between the transmitter-receiver 102 and the receiver 106 becomes possible.

The transmitter-receiver main body 103 has an amplifying section 201 amplifying and outputting a signal having been received by the first electrode 104, a filter (low-pass filter in the present embodiment) 202 through which a signal of predetermined frequency in an output signal of the amplifying section 201 passes, a heartbeat detecting section 203 detecting a heartbeat signal from an output signal of the filter 202, a first processor section 204 calculating a heartbeat number on the basis of the heartbeat signal having been detected in the heartbeat detecting section 203 to thereby output a heartbeat number data signal denoting the heartbeat number, a modulating section 205 modulating and outputting the heartbeat number data signal, and a data transmitting section 206 outputting the heartbeat number data signal having been modulated in the modulating section 205 from the electrode 104.

The filter 202 is a filter for removing a noise signal other than the heartbeat signal. Further, a frequency of a carrier signal that the modulating section 205 uses for modulating the heartbeat number data signal, i.e., a frequency of a carrier signal used in a communication between the transmitter-receiver 102 and the receiver 106, is set so as to differ from a frequency of the heartbeat signal. By this, there is constituted such that a signal communicated between the transmitter-receiver 102 and the receiver 106 and the heartbeat signal do not interfere. Incidentally, about a method of determining the frequency of the carrier signal, there is mentioned later.

The receiver main body 107 has an amplifying section 301 amplifying and outputting the heartbeat number data signal having been received by the third electrode 108, a demodulating section 302 demodulating an output signal of the amplifying section 301, a memory 304 as a storage means, a display section 305 as a display means, and a second processor section 303 which obtains a heartbeat number from the heartbeat number data signal having been demodulated by the demodulating section 302 to thereby store the heartbeat number to the memory 304, and which display-processes the heartbeat number to the display section 305.

In an ictometer system having been constituted like the above, in a case where the transmitter-receiver 102 is in a detecting mode, the amplifying section 201 amplifies and outputs the heartbeat signal having been received (detected) by the electrode 104. The filter 202 causes the signal of predetermined frequency in the output signal of the amplifying section 201 to pass and outputs it. The heartbeat detecting section 203 detects and outputs the heartbeat signal from the output signal of the filter 202. The processor section 204 calculates the heartbeat number on the basis of the heartbeat signal having been detected in the heartbeat detecting section 203.

Next, if the transmitter-receiver 102 becomes a transmitting mode, the processor section 204 outputs the heartbeat number data signal denoting the heartbeat number having been calculated on the basis of the heartbeat signal having been detected in the heartbeat detecting section 203 to the modulating section 205. The modulating section 205 modulates the heartbeat number data signal from the processor section 204 by the carrier signal of predetermined frequency, and outputs it. The data transmitting section 206 outputs the heartbeat number data signal having been modulated in the modulating section 205 from the electrode 104.

The heartbeat number data signal having been outputted from the electrode 104 is received by the third electrode 108 of the receiver 106 while passing the human body 101 (mainly, the chest part and the left arm).

The receiver main body 107 amplifies the heartbeat number data signal having been received by the electrode 108 in the amplifying section 301, and outputs it. The demodulating section 302 demodulates and outputs the heartbeat number data signal having been outputted from the amplifying section 301. The second processor section 303 obtains the heartbeat number from the heartbeat number data signal having been outputted from the demodulating section 302 to thereby store in order the heartbeat number to the memory 304 in order by which the heartbeat number data signal has been received, and displays the heartbeat number to the display part 305.

As having been mentioned above, the via-human-body information transmission system concerned with the embodiment of the present invention possesses the transmitter-receiver 102 as a first communications means, which ahs the two electrodes 104, 105 contacting with or capacitance-connected to the human body 101, and which is used while being attached to the human body 101, and the receiver 106 as a second communication means, which has the one electrode 108 contacting with or capacitance-connected to the human body 101 and the one electrode 109 having been disposed while being directed to the outside of the human body 101, and which is used while being attached to the human body 101.

Accordingly, since it becomes possible to constitute the distance, by which the capacitance connection is performed, shortly in some extent, there becomes such that a communication operation between the transmitter-receiver 102 and the receiver 106 is more stabilized.

Further, since the transmitter-receiver 102 and the receiver 106 are attached respectively to the chest and the arm and a signal path via the human body 101 is long, it becomes possible to reduce an influence by a person's posture and the like in comparison with a case where the transmission path by the capacitance connection is long.

Further, since the two electrodes 104, 105 of the transmitter-receiver 102 are used both for the electrode performing the transmission/reception of the signal and for the electrode detecting an organism information of the human body 101, the communication of the signal and the detection of the organism information become possible by a simple constitution. Further, it becomes unnecessary to newly provide a communicating circuit such as RF circuit, so that a great reduction in cost becomes possible.

Further, since there is constituted such that a carrier frequency of the transmitter-receiver 102 and a frequency of the organism information (e.g., the heartbeat signal) are different, there is brought about such an advantage that it becomes possible to suppress the interference between a transmission signal and an organism information signal.

Further, there is constituted such that the transmitter-receiver 102 is attached to the chest of the human body 101 and the receiver 106 is attached to the arm of the human body 101, so that such a constitution as to enlarge the distance between the transmitter-receiver 102 and the receiver 106 is possible.

Further, according to the transmitter-receiver 102 concerned with the embodiment of the present invention, since the above via-human-body information transmission system can be constructed and especially the electrodes are used both for the communication and for the organism information detection, there is bought about an advantage that it becomes possible to simplify the constitution.

Next, there is explained about the method of determining the carrier signal frequency of the transmitter-receiver 102.

FIG. 4 is an explanatory view when determining the carrier frequency of the transmitter-receiver 102 in the above embodiment. In FIG. 4, the same reference numeral is applied to the same portion as FIG. 1, and an antenna (not shown in the drawing) for measurement is disposed at a measuring point 111 having been separated from the transmitter-receiver 102 by a predetermined distance.

Further, FIG. 5 is a characteristic diagram showing a frequency characteristic of the embodiment, wherein a solid line denotes a reception intensity of a signal having been received from the transmitter-receiver 102 by the electrode 108 of the receiver 106, and a broken line denotes a reception intensity of a signal having been received by the antenna at the measuring point 111.

In FIG. 4 and FIG. 5, the receiver 106 receives a signal from the transmitter-receiver 102 mainly via the human body. On the other hand, at the measuring point 111, a signal having propagated in the air from the transmitter-receiver 102 is received by the antenna and measured. From viewpoints of well performing the transmission of the signal and reducing an unnecessary radiation, it is desirable that an intensity of the signal that the receiver 106 has received from the transmitter-receiver 102 is large, and the intensity at the measuring point 111 is rather small.

In the present embodiment, from such viewpoints, there is adapted such that there is used the carrier signal within a range from 5 MHZ that is a frequency at which the intensity of the signal that the receiver 106 has received from the transmitter-receiver 102 does not decrease and the intensity of the signal having been measured at the measuring point 111 greatly decreases to 45 MHz that is a frequency at which the intensity of the signal that the receiver 106 has received from the transmitter-receiver 102 starts to decrease and the intensity of the signal having been measured at the measuring point 111 does not so increase.

Like this, by making the frequency of the carrier signal that the modulating section 205 of the transmission/reception part 102 uses for modulating the heartbeat number data signal into the frequency within the range of 5 MHz-45 MHz, a good signal transmission becomes possible because the receiver 106 can receive the signal of a constant intensity from the transmitter-receiver 102, and it becomes possible to suppress low the unnecessary radiation.

Incidentally, in the above embodiment, a switching of the detecting mode and the transmitting mode in the transmitter-receiver 102 may be made so as to be automatically performed for every predetermined time, and further such various modifications are possible as to constitute such that a start or the switching of the detecting mode and the transmitting mode is operated by providing an operation part in the transmitter-receiver 102 and operating the operation part by a user.

Further, although the transmitter-receiver 102 has been given as an example of the first communication means, and the reception part 106 has been given as an example of the second communication means, various modifications are possible if it is a constitution capable of performing the communication between the first and second communication means such as to make the reception part 106 into a data logger exclusively for collecting the data, to make both of the first and second communication means into the transmitter-receiver, or to constitute so as to make the first and second communication means respectively into the receiver and the transmitter-receiver.

### INDUSTRIAL APPLICABILITY

As the via-human-body information transmission system, there can be applied not only to the ictometer but also to such various systems as to perform the communication between the plural communications means having been worn to the human body, besides an organism information measurement system such as pulsimeter and pedometer.

1. A via-human-body information transmission system performing an information transmission between plural communication means through a human body, comprising:
a first communication means which has first and second electrodes contacting with or capacitance-connected to the human body and which is used while being attached to the human body, and
a second communication means which has a third electrode contacting with or capacitance-connected to the human body and a fourth electrode having been disposed while being directed to an outside of the human body and which is used while being attached to the human body,
characterized in that a communication is performed between the first and second communication means by the facts that the first electrode and the third electrode are electrically connected through the human body, and that the second electrode and the fourth electrode are electrically connected by a capacitance connection.

2. A via-human-body information transmission system according to claim 1, characterized in that an organism information of the human body is transmitted between the first communication means and the second communication means, and the first and second electrodes of the first communication means are used both for an electrode detecting the organism information of the human body and for an electrode performing a transmission of the organism information.

3.-7. (canceled)

8. A transmitter-receiver characterized by having a filter through which a signal of a predetermined frequency in a signal having been received by an electrode contacting with or capacitance-connected to a human body passes and which outputs it, a heartbeat detecting section detecting a heartbeat signal from an output signal of the filter, a processor section calculating a heartbeat number on the basis of the heartbeat signal having been detected in the heartbeat detecting section to thereby output a heartbeat number data signal denoting the heartbeat number, a modulating section modulating and outputting the heartbeat number data signal, and a data transmitting section outputting the heartbeat number data signal having been modulated in the modulating section from the electrode.

9. A transmitter-receiver according to claim 8, characterized in that a frequency of a carrier signal that the modulating section uses for modulating the heartbeat number data signal is a frequency within a range of 5 MHz-45 MHz.
